(19) United States
(12) Patent Application Publication
(10) Pub. No.: US 2012/0080039 A1
(43) Pub. Date: Apr. 5, 2012

(54) AID FOR SHIMMING MAGNETIC DISCS

(75) Inventor: Ralf Siegert, Herten (DE)

(73) Assignee: SOPHONO, INC., Boulder, CO (US)

(21) Appl. No.: 13/256,571

(22) PCT Filed: Mar. 15, 2010
(86) PCT No.: PCT/DE2010/000281

§ 371 Date: Dec. 9, 2011

(30) Foreign Application Priority Data
Mar. 15, 2009 (DE) 20 2009 003 509.3
Apr. 15, 2009 (DE) 20 2009 005 475.6

(51) Int. Cl. A61B 19/00

(52) U.S. Cl. 128/845

(57) ABSTRACT

The aid is for lining magnetic plates borne on the head and is adapted to the implanted magnets in the form of a thin plate made of a ferromagnetic material.

Implantation of magnets has proved useful for fixing aids that are borne on the head, such as semi-implantable hearing aid components or prostheses. For a number of reasons the magnets are preferably employed as double magnets connected to one another. The complementary external components, e.g. especially the electronic components, the microphone, vibrator, and the battery for the hearing aid are attached using opposing magnets. These are typically embedded in plastic that is called the base plate. The implantable and external magnets are mutually attracted to one another and thereby fix the external retaining element, but also lead to a pressure load on the soft tissue that is disposed between the magnets, i.e. especially the skin, the subcutaneous fat tissue, and some of the musculature.

While on the one hand, the retention force should be as great as possible, on the other hand the load on the skin must not lead to circulation problems or pressure points. Since the forces that act on points of the soft tissue are inversely proportional to the surface on which the total magnetic force acts, the shape of the base plate must be adapted individually and optimally to the surface of the skin. Not only does this involve the geometric adaptation of the base plate to the surface shape of the skin in the region of the implanted magnets, it also involves taking its elasticity distribution into account. This is important because the implanted magnets have a different surface shape than the base plate and thus the resulting pressure distribution does not correspond to the skin surface.

The base plate is individually lined in order to obtain the best possible individual adaptation. Similar to direct dental lining methods, an autopolymer in a viscous state is applied to the base plate and the processing width is placed onto the skin of the temple in the area of the implanted magnets. The magnetic field adjusts the plate and draws it onto the skin so that the still viscous plastic is pressed away corresponding to the resulting individual force distribution. After it has hardened, the lined base plate is removed, the overhanging areas are milled off, and the supporting surface undergoes moderate machining.

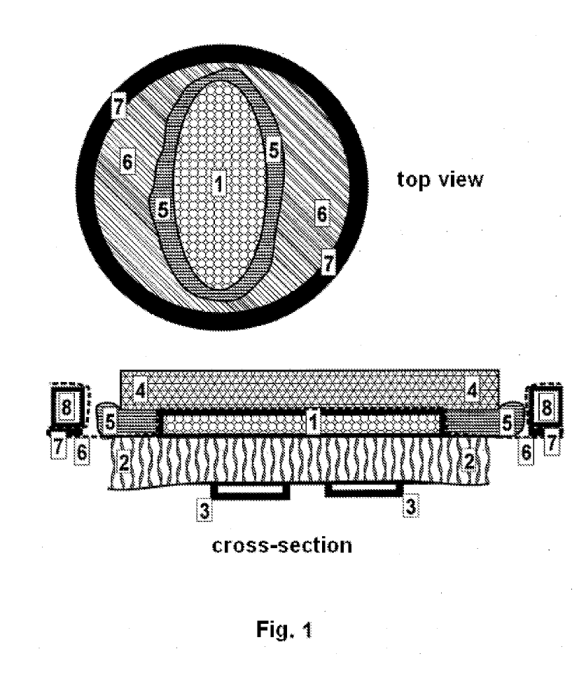

This—conventional—prior art leads to high geometrical congruence between the bottom of the base plate and the curvature of the skin over the implant. However, during this process the aforementioned different resilience in the soft tissue between the magnets is not compensated. Therefore at this point the following aid shall be introduced; one exemplary embodiment of it is depicted in FIG. 1:

A thin plate made of a ferromagnetic material (1) is placed onto the skin (2) over the implant comprising two magnets connected to one another (double magnet) (3) and fixed using its magnetic forces. The shape of this plate (1) corresponds approximately to the shape of the implant (3). Depending on the individual situation, however, it may also be advantageous when the metal plate (1) slightly overlaps the size of the implant (3) and is slightly beveled on the edge. This can prevent localized loads on the skin and can distribute the attracting forces more uniformly.

Now the lining is added in the manner described in the foregoing with the base plate (4) via the metal plate (1) and the surrounding skin (2). The metal plate and the hair should be insulated so that the autopolymer (5) that acts as the lining does not bind to them. To this end they are either lightly coated with an insulating film, e.g. made of Vaseline, or a plastic film (6) is stretched over the metal plate and the surrounding skin and hair. This film must be stretched in order to position it in the best possible way and with no folds with respect to the metal plate and the surrounding skin. To this end it is fixed in a solid frame. The latter preferably comprises a flat metal ring (7) about which the film is placed and on the back side of which it is fastened with a plurality of small magnets (8) adapted to the width of the ring. This metal ring (7) on which film is stretched is pressed over the plate (1) that is magnetically fixed on the implant and then the aforementioned lining is added. Once the plastic (5) has hardened, the lined base plate (4) is removed, the overhanging plastic is milled off, and its supporting surface undergoes moderate machining and is slightly smoothed without changing its shape, which is adapted to the individual.

Using this technology, the base plate is somewhat hollow in the regions of the highest pressure forces so that the force is more uniformly distributed onto the entire surface of the base plate and thus the risk of localized microcirculation problems and pressure points is reduced. As a rule, as suggested in the foregoing, its shape should be congruent with the shape of the implanted double magnets, but in certain cases it may be customized. In addition, metal plates of different size and thickness and beveled plates are available, and the thickness of these plates differs at the two ends. Using these latter types of plates it is possible to compensate both individual variances in skin thickness and also variable pressing forces onto the upper and lower magnets that are caused by the weight of the hearing aid.

The thickness and shape of the metal plate determine the depth of the hollow area and thus the extent of the pressure displacement onto the areas under the base plate (4) that are located adjacent to the implant. If the plate thickness is for instance 1 mm, the soft parts adjacent to the implant are pressed in 1 mm before the plate is supported across its entire surface. If the plate thickness is 2 mm, the soft parts outside of the implant are also correspondingly pressed 2 mm.

The selection of the plate strength and thus of the hollow area is a function of the individual anatomical situation after implantation. It is determined individually by:
the elevation of the implant over the bone, which itself is a function of
   the thickness of the available bone;
   the shape of the bone;
   the implant geometry; and,
   the technical aspects of the implantation, as well as
the thickness and elasticity of the skin over and
adjacent to the implant.

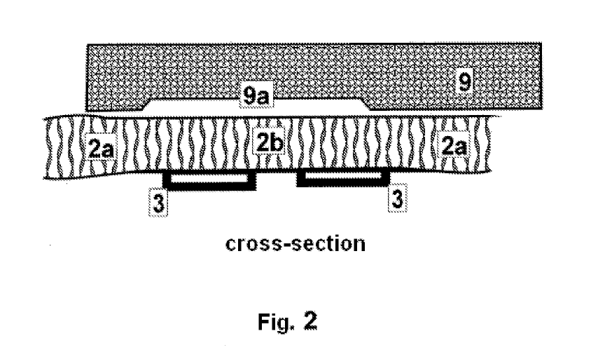

The aid depicted in FIG. 2 is proposed for determining the elasticity of the skin in vivo and thus the required plate thickness. It is a strip-shaped, solid plate (9) with a depression (9a) that is somewhat larger than the implanted magnet. A plurality of these measuring strips are available, each having a recess (9a) with a different depth. They are retained via the implanted magnets and pressed lightly into the skin adjacent to the implants (2a), i.e. until the skin becomes paler as an indication of decreased microcirculation. With this pressure the depression in the plate (9a) should just touch the skin over the magnet (2b). If there is still some empty space in this position, a plate (9) with a smaller depression (9a) is selected; on the other hand, if it presses against the skin (2b) until the skin becomes paler, it is exchanged for a plate (9) that has a larger depression (9a). The thickness of the metal plate ((1) in FIG. 1) is selected according to the depth of the depression ((9a) in FIG. 2).

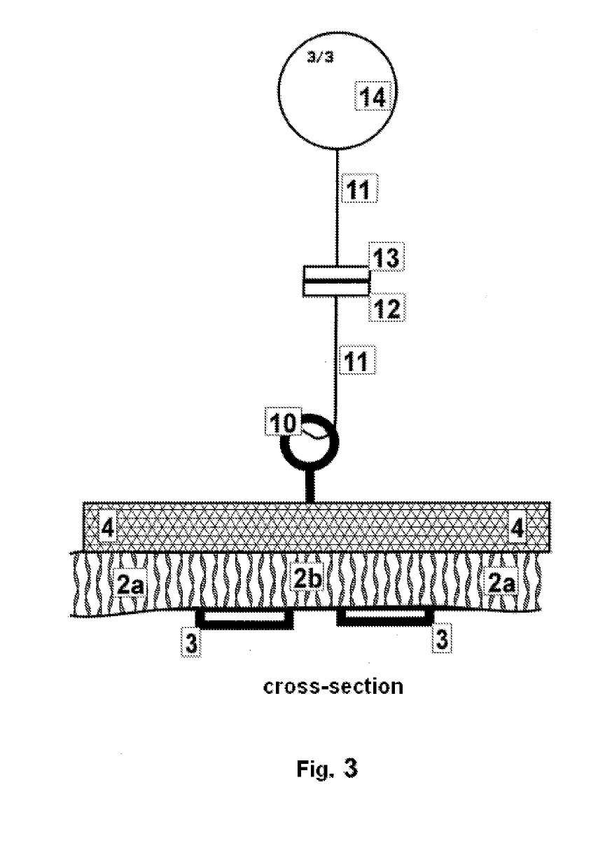

The total force with which the magnetic plate and the aid attached thereto (e.g. hearing aid or prosthesis) are retained depends both on the thickness and geometry of the implanted and the external magnets in the base plate and also on the distance between them. The approximate pressing forces can be selected by appropriately selecting the external magnets. Changes in the distance between the magnets may be precisely adjusted using the aforesaid lining and thereby make it possible to precisely adjust the pressing forces and their distribution. Various relatively small and easily handled force-displacement measurement systems are available for clinically measuring the total force; these systems register inter alia the maximum pulling force and thus the pressing force. They offer significantly greater measurement options than just determining the pulling force and are thus in many cases too complex and expensive for the routine inspection of a corresponding lining. Therefore the following simple aid for determining the approximate pulling force is presented; one exemplary embodiment of it is depicted in FIG. 3.

A small hook or eye is fixed in the base plate (4) using e.g. a thread. To perform the measurement, the simple pulling force measuring device is attached to this eye using a hook (10). Disposed on the hook is a tape (11), at the other end of which a small magnet (12) is attached. A second magnet (13) of about the same size is positioned against the first magnet; the poles of both magnets are such that they attract one another. A tape (11) is also disposed on the second magnet, and disposed at the other end of this tape is a loop or ring (14). The latter has a diameter such that it may be comfortably held by an index finger. The force with which the two magnets are attracted to one another is a function of their type, size, and geometry. They are selected such that they correspond to certain defined pulling forces. A plurality of these simple pulling force measuring apparatus with different magnetic strengths are available for precisely adjusting the pressing force of the base plate.

If one of these pulling force measuring devices is now attached to the base plate and pulled, at a certain pulling force either the base plate releases from the skin of the patient or the two magnets in the pulling apparatus release from one another. In the first instance the pressing force is less than the pulling force determined and registered in advance for the two magnets in the pulling apparatus and vice versa. By using different defined pulling apparatus it is possible to determine the pressure forces in steps, and there are different pulling apparatus available for the steps. In clinical practice increments of 0.5 N ranging from 1-5 N are adequate.

As an alternative to the two magnets (12 and 13) in the pulling apparatus, one of them may be replaced by a ferromagnetic plate (12). The force is then built up exclusively by the second magnet (13). The defined pulling forces are then varied and determined by exchanging this second magnet (13).

1. An aid for lining magnetic plates borne on the head, characterized in that the shape of a thin plate made of a ferromagnetic material is adapted to the implanted magnets.

2. Aid for supporting magnetic plates borne on the head in accordance with claim 1, characterized in that the metal plate slightly overlaps the size of the implant.

3. Aid for supporting magnetic plates borne on the head in accordance with claim 1, characterized in that the edge of the metal plate is beveled.

4. Aid for supporting magnetic plates borne on the head in accordance with claim 1, characterized in that different metal plates have different thicknesses.

5. Aid for supporting magnetic plates borne on the head in accordance with claim 1, characterized in that the individual metal plates have different thicknesses at different locations.

6. Aid for supporting magnetic plates borne on the head, characterized in that a strip-shaped, solid plate has a depression.

7. Aid for supporting magnetic plates borne on the head in accordance with claim 6, characterized in that the depression is adapted to the diameter of the implanted magnets and corresponds to them or is a few millimeters larger.

8. Aid for supporting magnetic plates borne on the head in accordance with claim 6, characterized in that the depression is present for estimating the soft tissue resilience in different dimensions (depths).

9. Aid for supporting magnetic plates borne on the head in accordance with claim 6, characterized in that the depressions of differing depth are disposed on an aid comprising one or a plurality of plates that are connected to one another.

10. Aid for supporting magnetic plates borne on the head in accordance with claim 6, characterized in that the recesses are labeled for identifying their dimensions, e.g. especially their depth.

11. Aid for supporting magnetic plates borne on the head, characterized in that a plurality of small magnets correspond to the width of a flat metal ring and thus on its back side fix a thin film that is stretched over the ring.

12. Aid for supporting magnetic plates borne on the head, characterized in that each of two magnets that have opposing poles and that attract one another are provided with tapes.

13. Aid for supporting magnetic plates borne on the head in accordance with claim 12, characterized in that hooks or rings are disposed at the ends of the tapes.

14. Aid for supporting magnetic plates borne on the head in accordance with claim 12, characterized in that one of the magnets is replaced by a ferromagnetic material.

15. Aid for supporting magnetic plates borne on the head in accordance with claim 12, characterized in that the magnets are present in various strengths and correspond to defined pulling forces for the two magnets or metal parts.
